(19) United States
(12) Patent Application Publication
(10) Pub. No.: US 2010/0280552 A1
(43) Pub. Date: Nov. 4, 2010

(54) SIDE CLICK CONNECTOR APPARATUS FOR CONNECTION OF SPINAL RODS

(76) Inventor: Choon Sung Lee, Seoul (KR)

Correspondence Address: BERLINER & ASSOCIATES, 555 WEST FIFTH STREET, 31ST FLOOR, LOS ANGELES, CA, 90013, US

(21) Appl. No.: 12/510,818

(22) Filed: Jul. 28, 2009

(30) Foreign Application Priority Data
May 4, 2009 (KR) 10-2009-0038954

(51) Int. Cl. A61B 17/70

(52) U.S. Cl. 606/250; 606/264

(57) ABSTRACT

Disclosed is a side click connector apparatus for connection of spinal rods, which connects a first spinal rod and a second spinal rod respectively attaching multiple pedicle screws and separately positioned from each other, and includes a connector body having a first mounting unit on which the first spinal rod is mounted, a second mounting unit on which the second spinal rod is mounted, a cutting part cut in the longitudinal direction of the corresponding spinal rod and formed in any one or both of the first mounting unit and the second mounting unit, more than one first retainer provided to be attached on and detached from the connector body to support the first spinal rod to the first mounting unit, and more than one second retainer provided to be attached on and detached from the connector body to support the second spinal rod to the second mounting unit.

### BACKGROUND OF THE INVENTION

### Field of the Invention

The present invention relates to a side click connector apparatus for connection of spinal rods and the associated surgical operating instruments. It is used to treat stenotic lesions or deformities developed with time in adjacent segments to the pre-existing spinal fixation system for degenerative spinal disorders (e.g. spinal stenosis, spondylolisthesis, or etc.) and spinal deformities (e.g. scoliosis, kyphosis, or etc.). Adjacent spinal lesions or deformities can be corrected or fixed using this invention. New rods are directly connected from the side to the pre-existing spinal fixation system and new pedicle screws without the need of interruption of the previous implants. Therefore, wide incision is not required with the current invention in revision spinal surgery.

### Description of the Related Art

Spinal arthrodesis (or referred to as spinal fusion) is a principal surgical method in treatment of degenerative spinal disorders or spinal deformities. Spinal arthrodesis is achieved using metal implants, such as pedicle screws and rods. In the past when the metal implants were not available, patients were inevitably immobilized for about 6 months with whole body cast, and the surgical results were poor. Spinal arthrodesis marked a revolutionary tuning point in the field of spinal surgery.

As more spinal fusions have been performed, however, several complications were found, with adjacent segment problem being the most important. Adjacent segment problem is a phenomenon that spinal stenosis or deformity develop in adjacent segments to the fused segments due to immobility of the fused segments and thus increased load concentration on the proximal or distal adjacent segments. Adjacent segment problem frequently requires revision surgery, in which the pre-existing metal implants must be removed. Therefore, revision surgery usually encompasses wide incision, long operation time, and high complication rate as well as an economic burden due to replacement of the previous implants with new ones.

The same problem may arise during post-operative follow-up of patients with spinal deformity such as scoliosis. If additional deformity developed in proximal or distal segments adjacent to the previously corrected and fused portion, revision surgery is required, in which removal of pre-existing metal implants is mandatory. It also has problems such as a heavy burden of complicated surgery and a high cost.

The present invention for a patent discloses a side click connector (or lateral connector or side connector) apparatus, which can be connected to a rod using a narrow space between pedicle screws during revision spinal surgery with the pre-existing internal metal fixtures in place. This side click connector will make the revision spinal surgery simple and easy, and reduce economic burden.

Further, in case that spinal arthrodesis is extended to the first sacral vertebra, pedicle screw fixation alone may be insufficient. If severe, pedicle screws can be loosened from the sacral vertebra or be broken. In this case, stronger fixation is achieved by additional fixation with iliac screws.

However, the currently available iliac screw fixation devices include several difficulties. This side click connector apparatus would facilitate insertion of iliac screws. Therefore, the side click connector apparatus of the present invention is valuable in the insertion of iliac screws.

### SUMMARY OF THE INVENTION

The purpose of the present invention is to provide a side click connector apparatus for connection of spinal rods, which facilitates the connection between rods of the existing spinal fixing apparatus and newly added rods, and has a strong fixation force after locking using set screws. It is easily connected to the rods with a small force from the side, and is not easily disconnected from the rods, but maintains excellent rotational and axial mobility of the rods after connection.

This side click connector apparatus for connection of spinal rods connect spinal rods of pre-existing metal implants (first spinal rods) attaching multiple pedicle screws (inserted at index surgery), and spinal rods used in revision surgery (second spinal rods) which is separately positioned from the first spinal rods. It has a first mounting unit for the first spinal rods, and a second mounting unit for the second spinal rods separated from the above first mounting unit. The first and second mounting unit include a connector body that has longitudinal grooves and cutting parts for the corresponding spinal rods; more than one first retainer supporting the first spinal rod to the first mounting unit, which can be attached to and detached from the connector body; more than one second retainer supporting the second spinal rod to the second mounting unit, which can be attached to and detached from the connector body.

The first and second retainers are bolts, and can be screw-connected to the connector body to support the first spinal rod and the second spinal rod mounted on the first mounting unit and the second mounting unit.

Additionally, the connector body includes a coupler that makes the connection of the first and second mounting units tilt and swivel with each other.

The coupler includes a spherical ball protruded from any one of the first mounting unit or the second mounting unit, and a ball bearing part indented from the other one of the first mounting unit or the second mounting unit to surround the ball and support it so that the first mounting unit and the second mounting unit can be tilted and swiveled with each other.

A passing-through hole may be formed in the connector body of the mounting unit which does not have a cutting part for a spinal rod.

The cutting part may be cut from the outer edge of the connector body, and have a smaller width than the outer diameter of the corresponding spinal rod. Thereby, the spinal rod is mounted on the mounting unit with a click sound, when the spinal rod passes through the cutting part, and the spinal rod mounted on the mounting unit is not easily separated from the cutting part.

### BRIEF DESCRIPTION OF THE DRAWINGS

The purposes, features and advantages of the present invention will be more clearly understood from the following detailed description taken in conjunction with the accompanying drawings, in which:

FIG. 1 is a perspective view of a side click connector apparatus for connection of spinal rods in accordance with a first embodiment of the present invention;

FIG. 2 is an exploded perspective view of an essential part of FIG. 1;

FIG. 3 is a longitudinal-sectional view of FIG. 1 illustrating the side click connector apparatus in a state in which respective retainers are separated from a connector body;

FIG. 4 is a longitudinal-sectional view of FIG. 1 illustrating the side click connector apparatus in a state in which respective retainers are mounted on a connector body;

FIG. 5 is a perspective view illustrating the side click connector apparatus in a state in which a first mounting unit of FIG. 1 is tilted and swiveled relative to a second mounting unit;

FIG. 6 is a perspective view of a side click connector apparatus for connection of spinal rods in accordance with a second embodiment of the present invention;

FIG. 7 is a longitudinal-sectional view of FIG. 6 illustrating the side click connector apparatus in a state in which respective retainers are mounted on a connector body;

FIG. 8 is an exploded perspective view of an essential part of a side click connector apparatus for connection of spinal rods in accordance with a third embodiment of the present invention;

FIG. 9 is a perspective view of a side click connector apparatus for connection of spinal rods in accordance with a fourth embodiment of the present invention;

FIG. 10 is an exploded perspective view of an essential part of a side click connector apparatus for connection of spinal rods in accordance with a fifth embodiment of the present invention;

FIGS. 11 and 12 are plan views respectively illustrating the side click connector apparatuses in accordance with the present invention in the used state;

FIG. 13 is an enlarged perspective view of the essential part of FIG. 10; and

FIG. 14 is an enlarged perspective view of the essential part of the side click connector apparatus in accordance with the present invention in the used state.

### DETAILED DESCRIPTION OF THE INVENTION

Now, preferred embodiments of the present invention will be described in detail with reference to the annexed drawings.

Prior to description, in the several embodiments of the present invention, some parts having substantially the same constructions are denoted by the same reference numerals even though they are depicted in different drawings. Further, all parts in a first embodiment will be exemplarily described, and only some parts in other embodiments, which substantially differ from those of the first embodiment, will be described. The first and second embodiments have the same concept and principle. However, the second embodiment illustrates a fundamental apparatus, which is more widely used than an apparatus of the first embodiment.

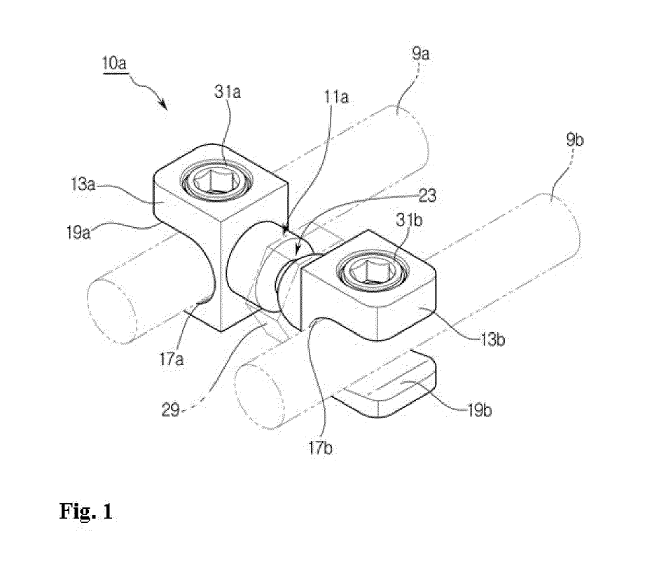

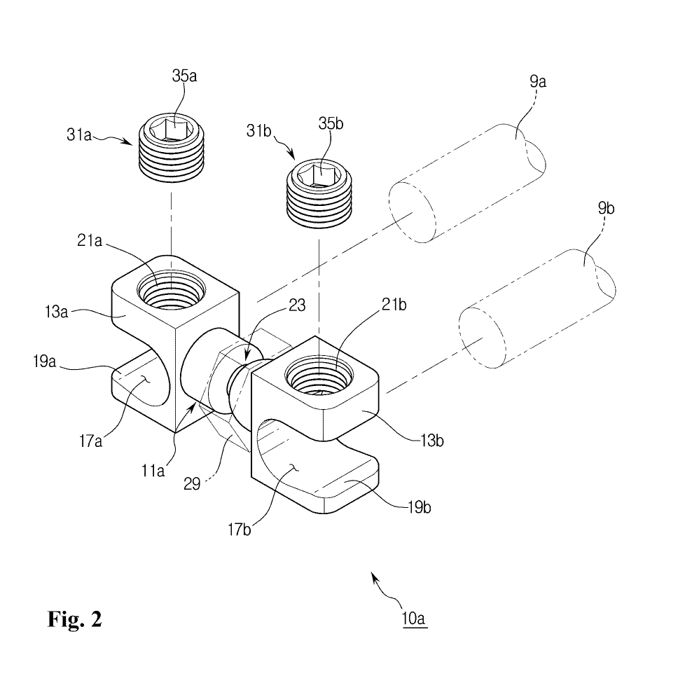

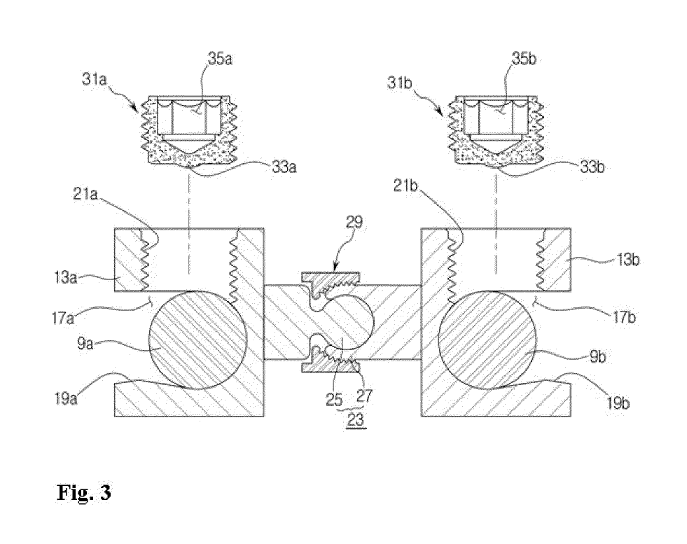

FIGS. 1 to 5 illustrate a side click connector apparatus 10a for connection of spinal rods in accordance with the first embodiment of the present invention. As shown in FIGS. 1 to 5, the side click connector apparatus 10a in accordance with the first embodiment connects a first spinal rod 9a and a second spinal rod 9b, which respectively connect multiple pedicle screws 1 and are separately positioned from each other.

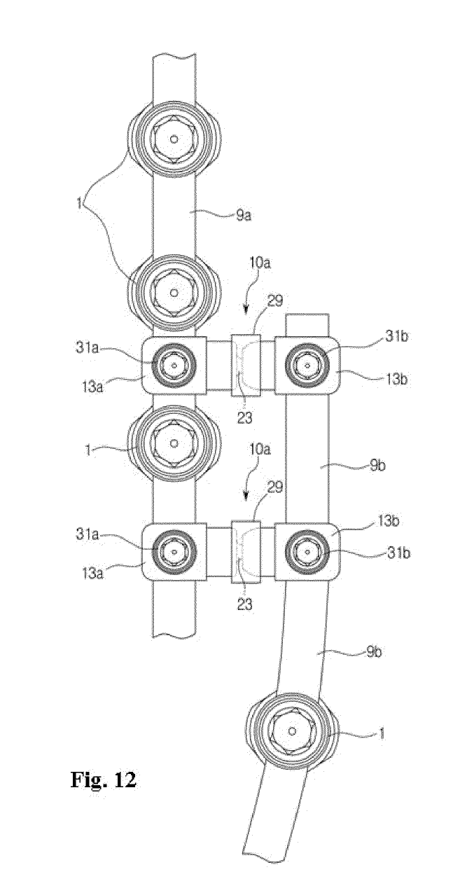

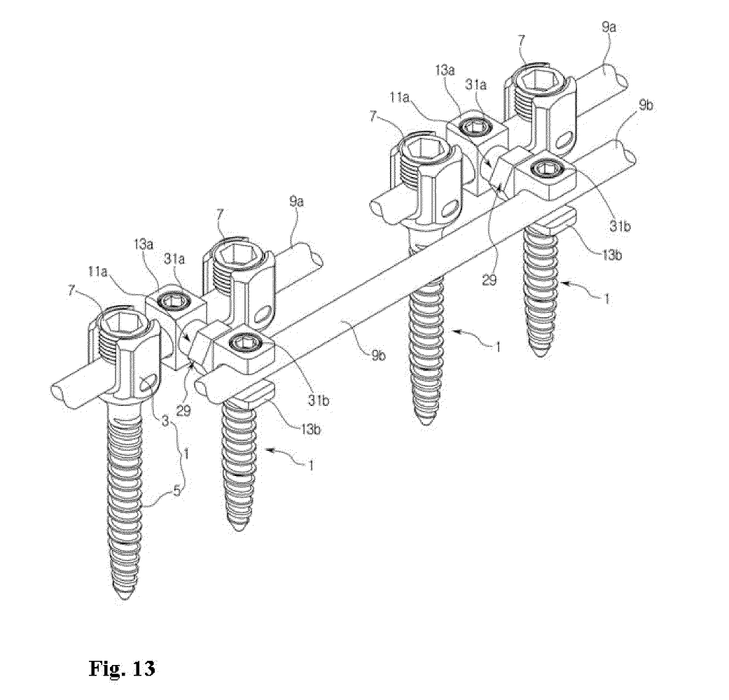

Prior to description of the side click connector apparatus 10a of the present invention, the structure of the pedicle screws 1 will be described in brief with reference to FIGS. 12 and 13. A pedicle screw 1 includes a head part 3 to receive the spinal rod 9a or 9b, and a screw part 5 longitudinally extended from one side of the head part 3 and fixed to the pedicle.

The spinal rods 9a and 9b are received in the head parts 3 of the pedicle screws 1, and are fixed to the pedicle screws 1 by set screws 7 respectively screw-connected to the head parts 3. A single or multiple pedicle screws 1 are connected to the spinal rods 9a and 9b.

The concept and principle of the first and second embodiments are the same except that first and second mounting units in the first embodiment are connected by a coupler and first and second mounting units in the second embodiment are connected by a connector body. Further, the second embodiment illustrates a fundamental apparatus, which is more widely used than that of the first embodiment.

As shown in FIGS. 1 to 5, the side click connector apparatus 10a in accordance with the first embodiment of the present invention includes a connector body 11a on which the first spinal rod 9a and the second spinal rod 9b are mounted, a first retainer 31a to support the first spinal rod 9a to the connector body 11a, and a second retainer 31b to support the second spinal rod 9b to the connector body 11a.

The connector body 11a includes a first mounting unit 13a on which the first spinal rod 9a is mounted, and a second mounting unit 13b on which the second spinal rod 9b is mounted, separated from the first mounting unit 13a. In this embodiment, reception grooves 17a and 17b receiving the first spinal rod 9a and the second spinal rod 9b are respectively formed on the first mounting unit 13a and the second mounting unit 13b in order to partially surround the circumferences of the first spinal rod 9a and the second spinal rod 9b. The first mounting unit 13a includes a cutting part 19a cut from the outer edge of the connector body 11a in the longitudinal direction of the first spinal rod 9a and communicated with the first reception groove 17a so that the first spinal rod 9a can enter the first reception groove 17a toward the second mounting unit 13b. Further, the second mounting unit 13b includes a cutting part 19b cut from the outer edge of the connector body 11a in the longitudinal direction of the second spinal rod 9b and communicated with the second reception groove 17b so that the second spinal rod 9b can enter the second reception groove 17b toward the first mounting unit 13a. Accordingly, the respective reception grooves 17a and 17b of the first mounting unit 13a and the second mounting unit 13b have a U-shaped section when viewed from the side.

The widths of the respective cutting parts 19a and 19b of the first mounting unit 13a and the second mounting unit 13b are smaller than the outer diameters of the corresponding spinal rods 9a and 9b. Therefore, the spinal rods 9a and 9b are received in the reception grooves 17a and 17b of the mounting units 13a and 13b with a click sound, when they pass through the cutting parts 19a and 19b. Then, the spinal rods 9a and 9b received in the reception grooves 17a and 17b are not easily separated from the reception grooves 17a and 17b by the cutting parts 19a and 19b.

A first screw hole 21a and a second screw hole 21b, with which the first retainer 31a and the second retainer 31b are respectively screw-connected, are formed on the upper surfaces of the first mounting unit 13a and the second mounting unit 13b.

The first retainer 31a is provided to be attached on and detached from the connector body 11a, and supports the first spinal rod 9a to the first reception groove 17a of the first mounting unit 13a.

The second retainer 31b is provided to be attached on and detached from the connector body 11a, and supports the second spinal rod 9b to the second reception groove 17b of the second mounting unit 13b.

The first retainer 31a and the second retainer 31b are bolts, and they are respectively screw-connected with the first screw hole 21a and the second screw hole 21b formed on the connector body 11a.

Elastic deformation parts 33a and 33b, which are elastically deformed in a direction opposite to the screw tightening of the respective retainers 31a and 31b with the application of pressure to the spinal rods 9a and 9b, are provided at the front ends of the first retainer 31a and the second retainer 31b facing the respective spinal rods 9a and 9b. Further, tool holding slots 35a and 35b indented to a designated depth to hold a tool, such as a wrench to connect and disconnect the respective retainers 31a and 31b to and from the screw holes 21a and 21b of the connector body 11a, are formed in the retainers 31a and 31b.

The tool holding slots 35a and 35b are indented to the designated depth in the longitudinal direction of the retainers 31a and 31b to allow the elastic deformation parts 33a and 33b to have an elastically deformable depth.

The side click connector apparatus 10a in accordance with the first embodiment of the present invention further includes a coupler 23 provided on the connector body 11a to interconnect the first mounting unit 13a and the second mounting unit 13b so that the first mounting unit 13a and the second mounting unit 13b can be tilted and swiveled with each other.

The coupler 23 includes a spherical ball 25 protruded from the end of the first mounting unit 13a, and a ball bearing part 27 indented from the end of the second mounting unit 13b to surround the ball 25 and support the ball 25 so that the first mounting unit 13a and the second mounting unit 13b can be tilted and swiveled with each other.

The ball bearing part 27 partially surrounds the ball 25 to prevent the ball 25 from being separated from the ball bearing part 27, and a sliding contact plane having a concave spherical shape approximately coinciding with the spherical shape of the ball 25 is formed on the inner surface of the ball bearing part 27.

The ball 25 and the ball bearing part 27 are fixed to each other by a fixing nut 29. That is, the fixing nut 29 is screw-connected with the ball bearing part 27 and also partially surrounds the end of the first mounting unit 13a.

Thereby, the ball 25 does not move within the ball bearing part 27.

Accordingly, the first mounting unit 13a can be freely tilted and swiveled relative to the second mounting unit 13b around the ball 25, as shown in FIG. 5, and thus a new spinal rod may be connected to the pre-existing spinal rod without the interruption of the previous spinal fixing apparatus and the additional skin incision. Further, the side click connector apparatus is connected to the spinal implants with a small force, and allows axial and rotational movement after connection so that it can make the rods fixed in proper positions.

By the above configuration, a process of connecting the first spinal rod 9a and the second spinal rod 9b, separately positioned from each other, with the use of the side click connector apparatus 10a in accordance with the first embodiment of the present invention will be described as follows. First, the first spinal rod 9a is inserted into the first reception groove 17a through the cutting part 19a of the first mounting unit 13a without interference with the adjacent pedicle screw 1.

Thereafter, the second spinal rod 9b is inserted into the second reception groove 17b through the cutting part 19b of the second mounting unit 13b.

At this time, the first mounting unit 13a is tilted and swiveled relative to the second mounting unit 13b by the coupler 23 according to the positions of the respective spinal rods 9a and 9b.

Thereafter, the first retainer 31a and the second retainer 31b are screw-connected with the first screw hole 21a and the second screw hole 21b so that the first spinal rod 9a and the second spinal rod 9b do not move in the first reception groove 17a of the first mounting unit 13a and the second reception groove 17b of the second mounting unit 13b.

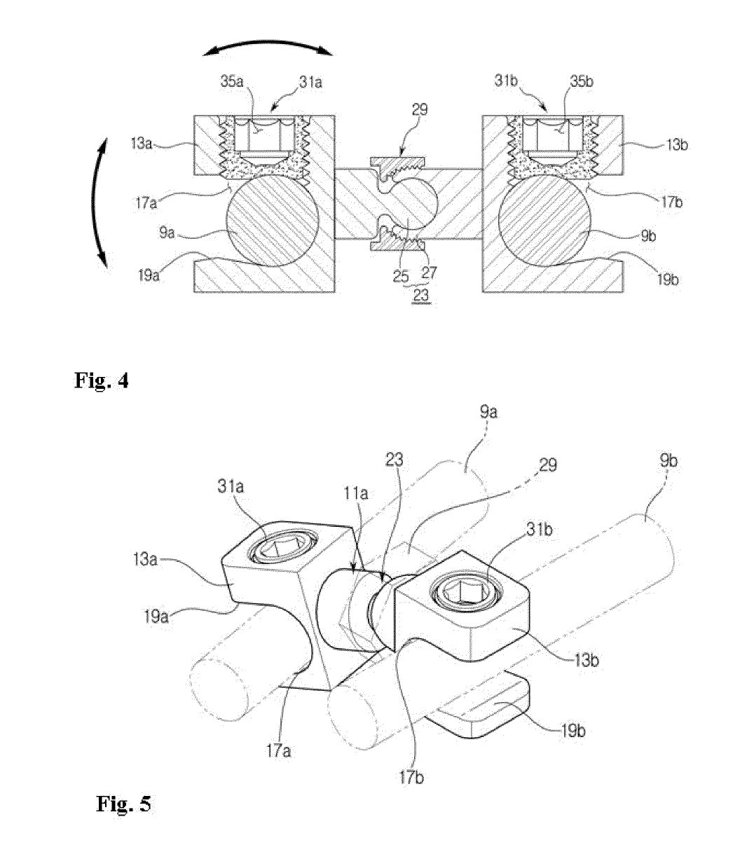

When the elastic deformation parts 33a and 33b of the respective retainers 31a and 31b apply pressure to the respective spinal rods 9a and 9b by at least a designated degree of the screw tightening force, as shown in FIG. 4, the elastic deformation parts 33a and 33b closely contact designated regions of the outer circumferences of the spinal rods 9a and 9b, and are elastically deformed in a direction opposite to the spinal rods 9a and 9b, i.e., in a direction opposite to the screw tightening direction of the retainers 31a and 31b. Thereby, the deformation of the screw holes 21a and 21b of the connector body 11a due to the tightening force of the retainers 31a and 31b is prevented, and thus the retainers 31a and 31b and the connector body 11a are stably screw-connected with a high fixation force to reduce the movements of the spinal rods 9a and 9b.

Further, the side click connector apparatus 10a in accordance with the first embodiment of the present invention may reduce steps to connect the pre-existing spinal fixing apparatus and a newly added spinal fixing apparatus, and be used at a region where the space between the pedicle screws 1 is narrow or there is a difference of heights between the spinal rods 9a and 9b.

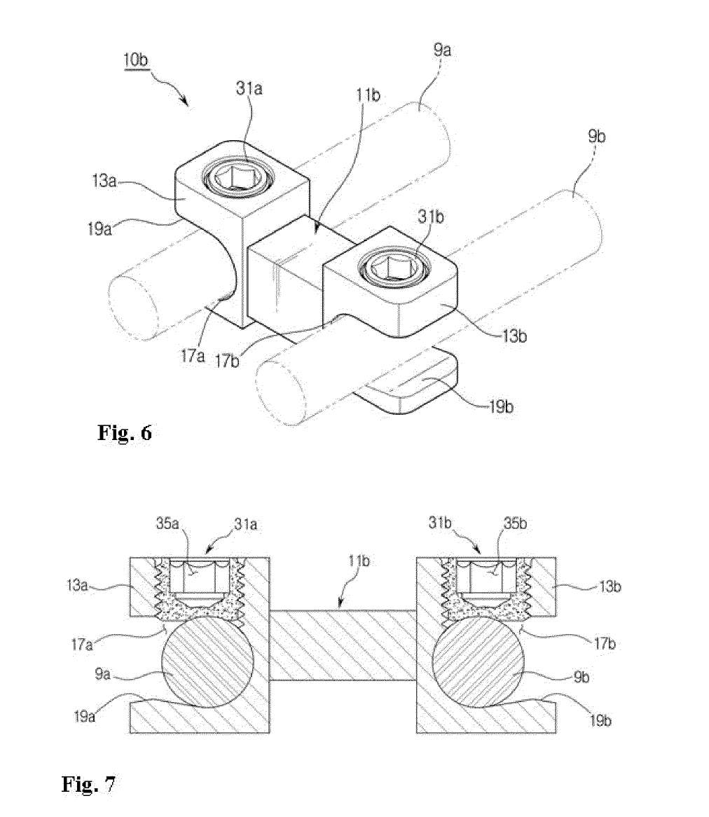

FIGS. 6 and 7 illustrate a side click connector apparatus 10b for connection of spinal rods in accordance with the second embodiment of the present invention. Differing from the above-described first embodiment, in the side click connector apparatus 10b in accordance with the second embodiment of the present invention, a first mounting unit 13a and a second mounting unit 13b of a connector body 11b are not connected by the coupler 23, but the first mounting unit 13a and the second mounting unit 13b are connected integrally with the connector body 11b.

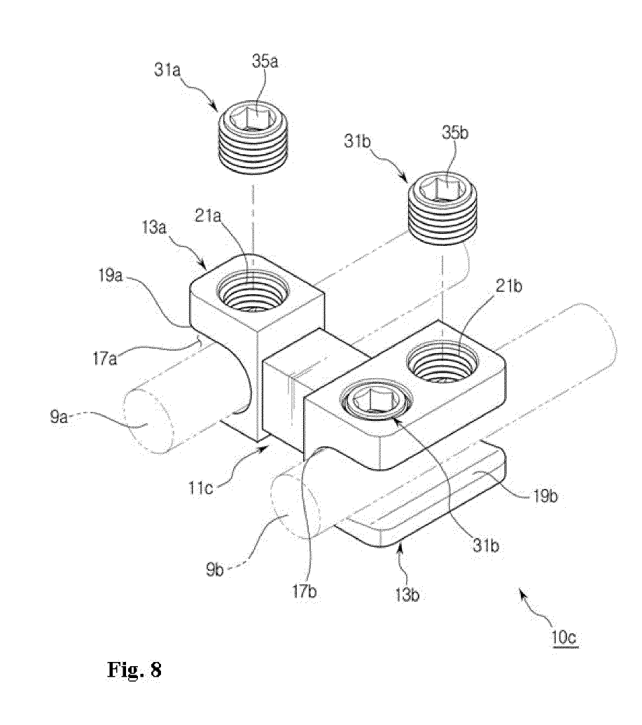

FIG. 8 illustrates a side click connector apparatus 10c for connection of spinal rods in accordance with a third embodiment of the present invention. Differing from the above-described second embodiment, in the side click connector apparatus 10c in accordance with the third embodiment of the present invention, two second retainers 31b are applied to a second mounting unit 13b, and thus the second retainers 31b are screw-connected to a connector body 11c with a strong fixation force and the movement of the second spinal rod 9b is more reduced.

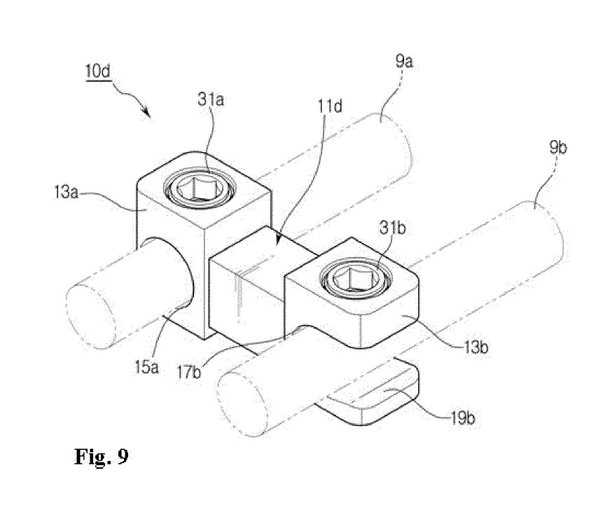

FIG. 9 illustrates a side click connector apparatus 10d for connection of spinal rods in accordance with a fourth embodiment of the present invention. Differing from the above-described second embodiment, in the side click connector apparatus 10d in accordance with the fourth embodiment of the present invention, a passing-through hole 15a to receive a first spinal rod 9a is formed in a first mounting unit 13a of a connector body 11d, and thus an end portion of the first spinal rod 9a is inserted into the first mounting unit 13a via the passing-through hole 15a and a middle portion of the second spinal rod 9b is received in and partially surrounded by a reception groove 17b of a second mounting unit 13.

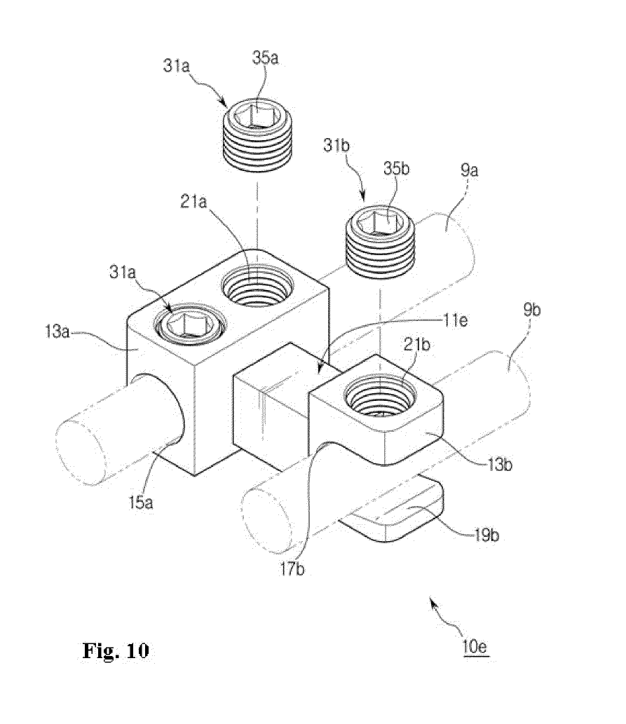

FIG. 10 illustrates a side click connector apparatus 10e for connection of spinal rods in accordance with a fifth embodiment of the present invention. Differing from the above-described fourth embodiment, in the side click connector apparatus 10e in accordance with the fifth embodiment of the present invention, two first retainers 31a are applied to a first mounting unit 13a, and thus the first retainers 31a are screw-connected to a connector body lie with a strong fixation force and the movement of the first spinal rod 9a is more reduced.

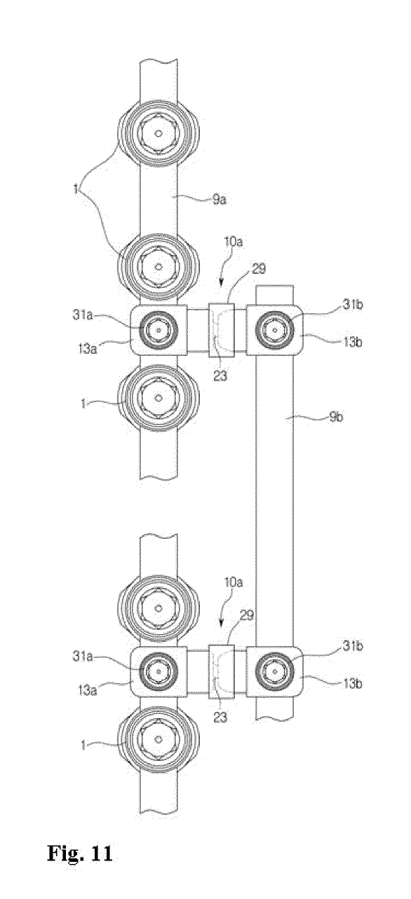

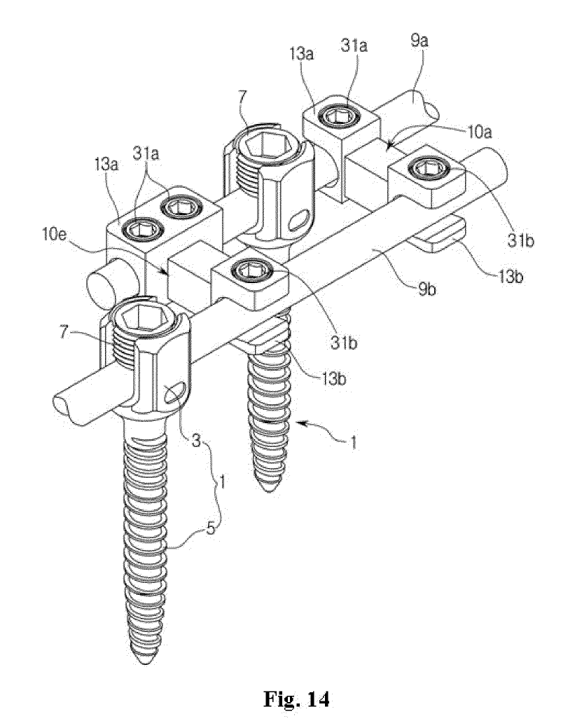

FIGS. 11 and 12 are plan views illustrating various connection states between the side click connector apparatuses in accordance with the present invention and a pair of spinal rods. FIGS. 13 and 14 are enlarged perspective views of the essential part of the connection states between the side click connector apparatuses in accordance with the present invention and a pair of spinal rods. As shown in FIGS. 11 to 14, the side click connector apparatus 10a in accordance with the first embodiment of the present invention is connected to a middle portion of a first spinal rod 9a having narrow intervals between pedicle screws 1 and an end portion of a second spinal rod 9b.

Further, FIG. 14 illustrates the side click connector apparatus 10e in accordance with the fifth embodiment of the present invention, which is connected to an end portion of a first spinal rod 9a and a middle portion of a second spinal rod 9b.

Therefore, the side click connector apparatuses in accordance with the various embodiments of the present invention may be selected according to positions of the respective spinal rods. That is, the mounting unit having a single retainer is mounted on the region of a spinal rod having narrow intervals between pedicle screws and mounting unit having more than two retainers is mounted on the region of a spinal rod having wide intervals between pedicle screws and requiring a strong fixation force, thereby maintaining an interval between the rods and adjusting pedicle screw angles and intervals during revision surgery.

Further, the side click connector apparatus in accordance with any one of the embodiments of the present invention easily connects a new spinal rod using a small amount of additional skin incision without the interruption of the pre-existing spinal fixing apparatus. Moreover, the side click connector apparatus in accordance with any one of the embodiments of the present invention is connected to the rods with a small force, and allows axial and rotational movement so that it can make the rods fixed in proper positions after connection.

The side click connector apparatus in accordance with any one of the embodiments of the present invention may be used in revision spinal surgery and iliac screw fixation.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A side click connector apparatus for connection of spinal rods, which connects a first spinal rod and a second spinal rod respectively attaching multiple pedicle screws and separately positioned from each other, comprising:
a connector body including a first mounting unit on which the first spinal rod is mounted, a second mounting unit on which the second spinal rod is mounted, and a cutting part cut in the longitudinal direction of the corresponding spinal rod and formed in any one or both of the first mounting unit and the second mounting unit so that the corresponding spinal rod comes into the corresponding one of the first mounting unit and the second mounting unit toward the other one of the first mounting unit and the second mounting unit;
at least one first retainer provided to be attached on or detached from the connector body to support the first spinal rod to the first mounting unit; and
at least one second retainer provided to be attached on or detached from the connector body to support the second spinal rod to the second mounting unit.

2. The side click connector apparatus according to claim 1, wherein the first retainer and the second retainer are bolts, and are screw-connected to the connector body to support the first spinal rod and the second spinal rod mounted on the first mounting unit and the second mounting unit.

3. The side click connector apparatus according to claim 1, further comprising a coupler provided on the connector body to connect the first mounting unit and the second mounting unit so that the first mounting unit and the second mounting unit can be tilted and swiveled with each other.

4. The side click connector apparatus according to claim 3, wherein the coupler includes:
a spherical ball protruded from any one of the first mounting unit and the second mounting unit; and
a ball bearing part indented from the other one of the first mounting unit and the second mounting unit to surround the ball and support the ball so that the first mounting unit and the second mounting unit can be tilted and swiveled with each other.

5. The side click connector apparatus according to claim 1, wherein a passing-through hole is formed in the other one of the first mounting unit and second mounting unit, in which the cutting part is not formed.

6. The side click connector apparatus according to claim 1, wherein the cutting part is cut from the outer edge of the connector body, and has a smaller width than the outer diameter of the corresponding spinal rod.
